(19) United States
(12) Patent Application Publication
(10) Pub. No.: US 2012/0143273 A1
(43) Pub. Date: Jun. 7, 2012

(54) IMPLANTABLE LEAD INCLUDING A SPARK GAP TO REDUCE HEATING IN MRI ENVIRONMENTS

(76) Inventors: Scott R. Stubbs, Maple Grove, MN (US); Arthur J. Foster, Centerville, MN (US)

(21) Appl. No.: 13/238,621

(22) Filed: Sep. 21, 2011

Related U.S. Application Data
(60) Provisional application No. 61/420,641, filed on Dec. 7, 2010.

(51) Int. Cl. A61N 1/39; A61N 1/05

(52) U.S. Cl. 607/5

(57) ABSTRACT

A medical device lead includes a proximal connector configured to couple the lead to a pulse generator, and an insulative lead body extending distally from the proximal connector. The first lead conductor is coupled to the proximal connector and extends through the lead body. The medical device lead also includes a distal defibrillation electrode. A first spark gap is connected between the first lead conductor and the distal defibrillation electrode and has a breakdown voltage that prevents transmission of magnetic resonance imaging (MRI) induced signals from the first lead conductor to the distal defibrillation electrode in an MRI environment and allows transmission of therapy signals to the distal defibrillation electrode.

### CROSS-REFERENCE TO RELATED APPLICATION

This application claims priority to Provisional Application No. 61/420,641, filed Dec. 7, 2010, which is herein incorporated by reference in its entirety.

### TECHNICAL FIELD

The present invention relates to implantable medical devices. More particularly, the present invention relates to a medical device lead including a spark gap to minimize transmission of MRI induced signals to shock electrodes.

### BACKGROUND

Magnetic resonance imaging (MRI) is a non-invasive imaging procedure that utilizes nuclear magnetic resonance techniques to render images within a patient's body. Typically, MRI systems employ the use of a magnetic coil having a magnetic field strength of between about 0.2 to 3 Teslas (T). During the procedure, the body tissue is briefly exposed to RF pulses of electromagnetic energy in a plane perpendicular to the magnetic field. The resultant electromagnetic energy from these pulses can be used to image the body tissue by measuring the relaxation properties of the excited atomic nuclei in the tissue.

During imaging, the electromagnetic radiation produced by the MRI system may be picked up by implantable device leads used in implantable medical devices such as pacemakers or cardiac defibrillators. This energy may be transferred through the lead to the electrode in contact with the tissue, which may lead to elevated temperatures at the point of contact. The degree of tissue heating is typically related to factors such as the length of the lead, the conductivity or impedance of the lead, and the surface area of the lead electrodes. Exposure to a magnetic field may also induce an undesired voltage on the lead.

### SUMMARY

Discussed herein are various components for implantable medical electrical leads including a spark gap that prevents transmission of MRI induced current to defibrillation electrodes, as well as medical electrical leads including such components.

In Example 1, a medical device lead includes a proximal connector configured to couple the lead to a pulse generator, and an insulative lead body extending distally from the proximal connector. The first lead conductor is coupled to the proximal connector and extends through the lead body. The medical device lead also includes a distal defibrillation electrode. A first spark gap is connected between the first lead conductor and the distal defibrillation electrode and has a breakdown voltage that prevents transmission of magnetic resonance imaging (MRI) induced signals from the first lead conductor to the distal defibrillation electrode in an MRI environment and allows transmission of therapy signals to the distal defibrillation electrode.

In Example 2, the medical device lead according to Example 1, and further comprising a second lead conductor coupled to the proximal connector and extending through the lead body, a proximal defibrillation electrode, and a second spark gap connected between the second lead conductor and the proximal defibrillation electrode, the second spark gap having a breakdown voltage that prevents transmission of MRI induced signals from the second lead conductor to the proximal defibrillation electrode in an MRI environment and allows transmission of a therapy signal to the proximal defibrillation electrode.

In Example 3, the medical device lead according to either Example 1 or 2, and further comprising one or more additional spark gaps connected along the first lead conductor to break the first lead conductor into segments each having lengths that are non-resonant with RF fields in the MRI environment.

In Example 4, the medical device lead according to any of Examples 1-3, wherein breakdown voltage is between about 100 volts (V) and about 150 V.

In Example 5, the medical device lead according to any of Examples 1-4, wherein, at voltages below the breakdown voltage, the first spark gap has a resistance greater than about 1,000 megaohm (MΩ) and a capacitance in the range of about 1.0 picofarad (pF) to about 5.0 pF.

In Example 6, the medical device lead according to any of Examples 1-5, wherein the first spark gap comprises two electrodes, and wherein the breakdown voltage is a function of a distance between the electrodes.

In Example 7, a medical device includes a pulse generator and a lead. The lead includes a proximal connector that couples the lead to the pulse generator, and an insulative lead body extending distally from the proximal connector. A first lead conductor is coupled to the proximal connector and extends through the lead body. The lead further includes a distal defibrillation electrode and a first spark gap connected between the first lead conductor and the distal defibrillation electrode. The first spark gap has a breakdown voltage that prevents transmission of MRI induced signals from the first lead conductor to the distal defibrillation electrode in an MRI environment and allows transmission of therapy signals to the distal defibrillation electrode.

In Example 8, the medical device according to Example 7, wherein, to deliver the therapy signal, the pulse generator is programmed to deliver a breakdown signal to the spark gap to establish current flow between the first lead conductor and the distal defibrillation electrode and subsequently provide the therapy signal.

In Example 9, the medical device according to either Example 7 or 8, wherein the pulse generator is programmed to disable transmission of therapy signals to the distal defibrillation electrode in the MRI environment.

In Example 10, the medical device according to any of Examples 7-9, and further comprising a second lead conductor coupled to the proximal connector and extending through the lead body, a proximal defibrillation electrode, and a second spark gap connected between the second lead conductor and the proximal defibrillation electrode, the second spark gap having a breakdown voltage that prevents transmission of MRI induced signals from the second lead conductor to the proximal defibrillation electrode in an MRI environment and allows transmission of a therapy signal to the proximal defibrillation electrode.

In Example 11, the medical device according to any of Examples 7-10, and further comprising one or more additional spark gaps connected along the first lead conductor to break the first lead conductor into segments each having lengths that are non-resonant with RF fields in the MRI environment.

In Example 12, the medical device according to any of Examples 7-11, wherein breakdown voltage is between about 100 V and about 150 V.

In Example 13, the medical device according to any of Examples 7-12, wherein, at voltages below the breakdown voltage, the first spark gap has a resistance greater than about 1,000 MΩ and a capacitance in the range of about 1.0 pF to about 5.0 pF.

In Example 14, the medical device according to any of Examples 7-13, wherein the first spark gap comprises two electrodes, and wherein the breakdown voltage is a function of a distance between the electrodes.

In Example 15, a medical device lead includes a proximal connector configured to couple the lead to a pulse generator, and an insulative lead body extending distally from the proximal connector. One or more low voltage conductors are coupled to the proximal connector and extend through the lead body. One or more pacing/sensing electrodes are each coupled to one of the one or more low voltage conductors. A proximal defibrillation conductor and distal defibrillation conductor are coupled to the proximal connector and extend through the lead body. The medical device lead further includes a proximal defibrillation electrode and a distal defibrillation electrode. A first spark gap is connected between the first lead conductor and the distal defibrillation electrode. The first spark gap having a breakdown voltage that prevents transmission of MRI induced signals from the first lead conductor to the distal defibrillation electrode in an MRI environment and allows transmission of therapy signals to the distal defibrillation electrode.

In Example 16, the medical device lead according to Example 15, and further comprising one or more additional spark gaps connected along the first lead conductor to break the first lead conductor into segments each having lengths that are non-resonant with RF fields in the MRI environment.

In Example 17, the medical device lead according to either Example 15 or 16, wherein breakdown voltage is between about 100 V and about 150 V.

In Example 18, the medical device lead according to any of Examples 15-17, wherein, at voltages below the breakdown voltage, the first spark gap has a resistance greater than about 1,000 MΩ and a capacitance in the range of about 1.0 pF to about 5.0 pF.

In Example 19, the medical device lead according to any of Examples 15-18, wherein the first spark gap comprises two electrodes, and wherein the breakdown voltage is a function of a distance between the electrodes.

In Example 20, the medical device lead according to any of Examples 15-19, and further comprising a second spark gap connected between the proximal defibrillation conductor and the proximal defibrillation electrode, the second spark gap having a breakdown voltage that prevents transmission of MRI induced signals from the proximal defibrillation conductor to the proximal defibrillation electrode in an MRI environment and allows transmission of a therapy signal to the proximal defibrillation electrode.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a cardiac rhythm management (CRM) system including a pulse generator and a lead implanted in a patient's heart according to an embodiment of the present invention.

FIG. 2 is a block diagram of portion of the CRM system illustrated in FIG. 1 including one or spark gaps connected between the lead conductors and defibrillation electrodes.

FIG. 3 is a cross-sectional view of an embodiment of a spark gap suitable for reducing transmission of MRI induced currents from the lead conductors to the defibrillation electrodes.

FIG. 4 is a schematic of a lead including a spark gap connected to a pulse generator.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

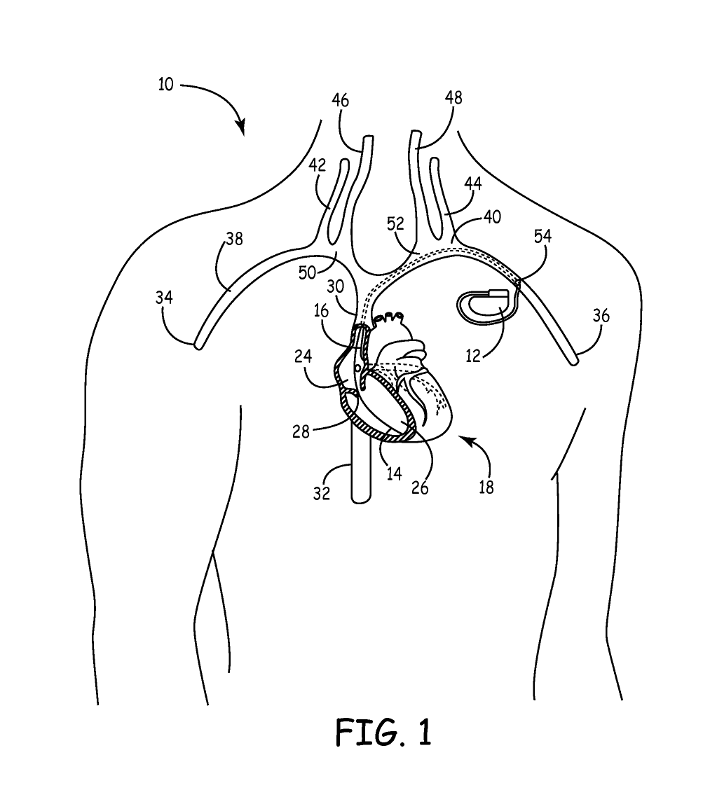

FIG. 1 is a schematic view of a cardiac rhythm management (CRM) system 10 according to an embodiment of the present invention. As shown in FIG. 1, the CRM system 10 includes a pulse generator 12 coupled to a plurality of leads 14, 16 deployed in a patient's heart 18. As further shown in FIG. 1, the heart 18 includes a right atrium 24 and a right ventricle 26 separated by a tricuspid valve 28. During normal operation of the heart 18, deoxygenated blood is fed into the right atrium 24 through the superior vena cava 30 and the inferior vena cava 32. The major veins supplying blood to the superior vena cava 30 include the right and left axillary veins 34 and 36, which flow into the right and left subclavian veins 38 and 40. The right and left external jugular 42 and 44, along with the right and left internal jugular 46 and 48, join the right and left subclavian veins 38 and 40 to form the right and left brachiocephalic veins 50 and 52, which in turn combine to flow into the superior vena cava 30.

The leads 14, 16 operate to convey electrical signals and stimuli between the heart 18 and the pulse generator 12. In the illustrated embodiment, the lead 14 is implanted in the right ventricle 26, and the lead 16 is implanted in the right atrium 24. In other embodiments, the CRM system 10 may include additional leads, e.g., a lead extending into a coronary vein for stimulating the left ventricle in a bi-ventricular pacing or cardiac resynchronization therapy system. As shown, the leads 14, 16 enter the vascular system through a vascular entry site 54 formed in the wall of the left subclavian vein 40, extend through the left brachiocephalic vein 52 and the superior vena cava 30, and are implanted in the right ventricle 26 and right atrium 24, respectively. In other embodiments of the present disclosure, the leads 14, 16 may enter the vascular system through the right subclavian vein 38, the left axillary vein 36, the left external jugular 44, the left internal jugular 48, or the left brachiocephalic vein 52.

The pulse generator 12 is typically implanted subcutaneously within an implantation location or pocket in the patient's chest or abdomen. The pulse generator 12 may be an implantable medical device known in the art or later developed, for delivering an electrical therapeutic stimulus to the patient. In various embodiments, the pulse generator 12 is a pacemaker, an implantable cardiac defibrillator, and/or includes both stimulation and defibrillation capabilities. The portion of the leads 14, 16 extending from the pulse generator 12 to the vascular entry site 54 are also located subcutaneously or submuscularly. The leads 14, 16 are each connected to the pulse generator 12 via proximal connectors. Any excess lead length, i.e., length beyond that needed to reach from the pulse generator 12 location to the desired endocardial or epicardial implantation site, is generally coiled up in the subcutaneous pocket near the pulse generator 12.

The electrical signals and stimuli conveyed by the pulse generator 12 are carried to electrodes at the distal ends of leads 14, 16 by one or more conductors extending through the leads 14, 16. The one or more conductors are each electrically coupled to a connector suitable for interfacing with the pulse generator 12 at the proximal end of the leads 14, 16 and to one or more electrodes at the distal end. In a magnetic resonance imaging (MRI) environment, the electromagnetic radiation produced by the MRI system may be picked up by conductors of the leads 14, 16. This energy may be transferred through the leads 14, 16 to the electrode in contact with the tissue, which may lead to elevated temperatures at the point of contact. The present invention relates to the use of a spark gap or surge arrester connected between the conductor and electrode to prevent transmission of MRI induced signals from the conductors to the electrodes. The spark gap has a breakdown voltage such that MRI induced signals are not transmitted across the gap, while therapy signals intended for the electrodes exceed the breakdown voltage and are transmitted across the gap to the electrodes.

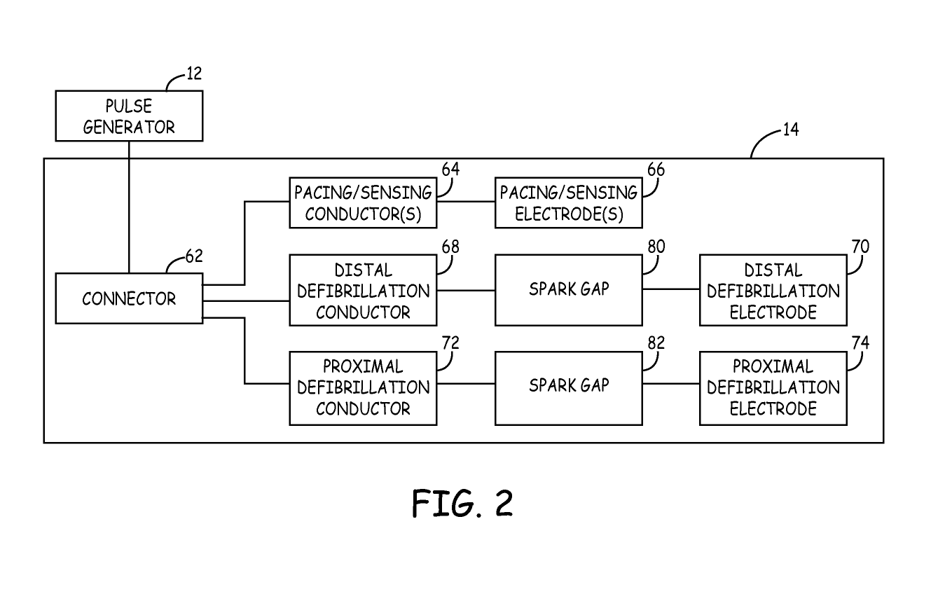

FIG. 2 is a block diagram of a lead 14 that may be suitable for use with the CRM system 10 shown in FIG. 1. While the lead 14 is shown, the lead 16 may have a similar construction. The lead 14, which is connected to the pulse generator 12, includes a proximal connector 62, one or more pacing/sensing conductors 64, one or more pacing/sensing electrodes 66, a distal defibrillation conductor 68, a distal defibrillation electrode 70, a proximal defibrillation conductor 72, and a proximal defibrillation electrode 74. The lead 14 also includes a spark gap 80 connected between the distal defibrillation conductor 68 and distal defibrillation electrode 70. The lead 14 may also include a spark gap 82 connected between the proximal defibrillation conductor 72 and proximal defibrillation electrode 74.

The pacing/sensing conductors 64, distal defibrillation conductor 68, and proximal defibrillation conductor 72 are connected to the proximal connector 62. The conductors 64, 68, and 72 extend distally through one or more lumens in an insulative lead body. In some embodiments, the pacing/sensing conductors 64, distal defibrillation conductor 68, and proximal defibrillation conductor 72 extend through the lead body in separate lumens. In other embodiments, some or all of the pacing/sensing conductors 64, distal defibrillation conductor 68, and proximal defibrillation conductor 72 extend through the same lumen in the lead body.

The pacing/sensing conductors 64 may include one or more conductive coils that extend coaxially or co-radially though the lead body. In some embodiments, the pacing/sensing conductors 64 comprise inner and outer co-axial conductors that each connect to one of the pacing/sensing electrodes 66 at or near the distal end of the lead 14. The connector 62 thus electrically connects the pacing/sensing electrodes 66 to the pulse generator 12. The signals carried by the pacing/sensing conductors 64 may be independently controlled by the pulse generator 12 such that different signals may be delivered to and/or received from each of the pacing/sensing electrodes 66. In some embodiments, the pacing/sensing electrodes 66 include a ring electrode and/or a tip electrode. In some embodiments, the pacing/sensing electrodes 66 include platinum or titanium coated with a combination of iridium oxide (IrOx), titanium/nickel (Ti/Ni), black platinum (Pt black), or tantalum oxide (TaO). The pacing/sensing electrodes 66 may be located near a distal end portion of the lead 14. In alternative embodiments, the pacing/sensing electrodes 66 are located elsewhere on the lead 14. The pacing/sensing conductors 64 and pacing/sensing electrodes 66 combine to form the low voltage pacing/sensing portion of the lead 14.

The distal defibrillation conductor 68 and proximal defibrillation conductor 72 may extend parallel to the pacing/sensing conductors 64 through the lead 14. In some embodiments, the distal defibrillation conductor 68 and/or proximal defibrillation conductor 72 comprise a conductive cable. In other embodiments, the distal defibrillation conductor 68 and/or proximal defibrillation conductor 72 comprise a conductive coil. The distal defibrillation conductor 68 and proximal defibrillation conductor 72 are configured to carry high energy signals to the distal defibrillation electrode 70 and the proximal defibrillation electrode 74, respectively. In some embodiments, the distal defibrillation conductor 68 and proximal defibrillation conductor 72 are each electrically coupled to one or more capacitors that develop the high energy charge (e.g., 40 Joules, 750 volts (V)). The pulse generator 12 is programmed to release the energy in the capacitors to the conductors 68, 72 when a cardiac event (e.g., arrhythmia, ventricular fibrillation, ventricular tachycardia) is sensed, such as by the pacing/sensing electrodes 66. In some embodiments, one or both of the defibrillation electrodes 70, 74 are also configured to provide sensing signals to the pulse generator 12.

In an MRI environment, the radio frequency (RF) fields can induce a current in the conductive elements of the lead 14. This current may then be dissipated at the point of contact between the lead electrodes and adjacent tissue, resulting in elevated temperatures in the tissue. To reduce the RF current that is transmitted to the distal defibrillation electrode 70 by the distal defibrillation conductor 68, the spark gap 80 may be connected between the distal defibrillation electrode 70 and the distal defibrillation conductor 68. The spark gap 80 is configured to disconnect the distal defibrillation electrode 70 from the distal defibrillation conductor 68 when a high voltage signal is not being delivered to the distal defibrillation electrode 70, but allows transmission of therapy signals to the distal defibrillation electrode 70. This prevents MRI induced signals that are received on the distal defibrillation conductor 68 from being transmitted to the distal defibrillation electrode 70, thereby preventing or minimizing heating of the distal defibrillation electrode 70.

In some embodiments, the spark gap 80 is also connected between the proximal defibrillation electrode 74 and the proximal defibrillation conductor 72 to reduce the RF current that is transmitted to the proximal defibrillation electrode 74 by the proximal defibrillation conductor 72. In other embodiments, for example in which the length of the distal defibrillation conductor 68 or proximal defibrillation conductor 72 is not resonant with the RF fields in the MRI environment, the spark gap 80 or 82 is not included in the lead 14, and the defibrillation conductor 68, 72 is connected directly to the defibrillation electrode 70, 74, respectively.

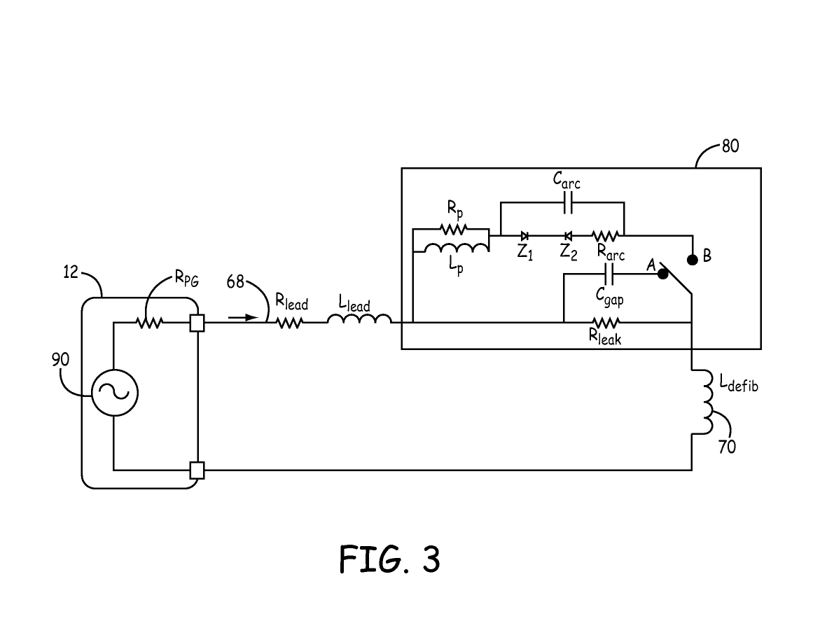

FIG. 3 is a schematic of a circuit including the pulse generator 12, distal defibrillation conductor 68, one or more shock gaps 80, and the distal defibrillation electrode 70. The circuit shown in FIG. 3 is derived, in part, from Christophe Basso, “SPICE Model Simulates Spark-Gap Arrestor,” Electronics Design, Strategy, and News (EDN) (Jul. 3, 1997), which incorporated herein by reference in its entirety. In embodiments including the spark gap 82, a similar schematic may be drawn includes the pulse generator 12, proximal defibrillation conductor 72, the spark gap 82, and the proximal defibrillation electrode 74. The pulse generator 12 is represented in the schematic by a current source 90 and a series resistance R_{PG}. The distal defibrillation conductor 68 has a lead resistance R_{lead} and a lead inductance L_{lead}, and the distal defibrillation electrode 70 has an inductance L_{defib}.

The conducting state of the spark gap 80 depends on the voltage drop across the spark gap 80. In particular, when the voltage across the spark gap 80 is less than the breakdown voltage of the spark gap 80, current does not flow across the spark gap 80. On the other hand, when the voltage across the spark gap 80 equals or exceeds the breakdown voltage of the spark gap 80, an arc forms across the spark gap 80, which allows current to flow through the spark gap 80. The breakdown voltage of the spark gap 80 is selected such that MRI induced voltage on the distal defibrillation conductor 68 does not exceed the breakdown voltage, while therapy delivered on the distal defibrillation conductor 68 does exceed the breakdown voltage. The breakdown voltage of the spark gap 80 is a function of various mechanical properties of the spark gap 80, including the size of the spark gap electrode plates, the distance between the plates, the media between the plates, the plate material, and the plate shape. In some embodiments, the breakdown voltage of the spark gap 80 is between about 100 V and about 150 V. In FIG. 3, the conducting states of the spark gap 80 are represented by a switch, with the switch connected to node A in the non-conducting state and to node B in the conducting state.

When therapy is not being delivered to the distal defibrillation conductor 68 and/or when the lead 14 is in an MRI environment, the spark gap 80 is modeled as a capacitance C_{gap} in parallel with a leakage resistance Rₗₑₐₖ. The capacitance C_{gap} is small to minimize RF current transmission to the distal defibrillation electrode 70, since the impedance of a capacitor is inversely proportional to the capacitance. In some embodiments, the spark gap 80 is designed to have a capacitance C_{gap} of about 1.0 picofarad (pF) to about 5.0 pF. The leakage resistance Rₗₑₐₖ is typically large in the spark gap 80. In some embodiments, the leakage resistance Rₗₑₐₖ is greater than about 1,000 megaohms (MΩ).

If the voltage across the spark gap 80 is increased sufficiently (including sufficient slew rate), the media (e.g., argon or neon) between the electrode plates of the spark gap 80 forms a plasma. In the plasma phase, the spark gap 80 transitions its behavior by appearing to drop the impedance between the plates. Conduction filaments form between the plates and a small current begins to flow between them. If the current through the spark gap 80 continues to rise due to the falling impedance, the spark gap 80 will transition quickly from plasma phase to arc phase. The time to transition from initial phase to plasma phase to arc phase when the input voltage slews sufficiently fast occurs in approximately 10 ns. In the arc phase, the spark gap 80 conducts current, which is represented by the switch connecting to node B in FIG. 3.

In the conducting state, the conducting spark gap 80 is represented by a capacitance C_{arc} connected in parallel with series connected Zener diodes Z₁ and Z₂ and gap resistance R_{arc}. The spark gap 80 also includes a parallel connected parasitic resistance Rₚ and parasitic inductance Lₚ from the lead wires of the spark gap 80. The lead wire parasitics are connected in series with the conducting spark gap 80.

When the arc across the spark gap 80 is generated, the arc will continue as long as sufficient current is available to keep the conduction channel open in the arc. The voltage across the spark gap 80 can fall below the threshold for initiating the arc, but the arc will remain in effect as long as sufficient current flows across the spark gap 80.

For therapy delivery, the pulse generator 12 charges the capacitor(s) connected to the distal defibrillation conductor 68 to a level that exceeds the breakdown voltage of the spark gap 80. When the therapy signal is delivered, the spark gap 80 quickly transitions to its arc phase and delivers the therapy signal to the distal defibrillation electrode 70. The breakdown voltage is fixed in the spark gap 80, and establishes the minimum energy of the therapy signal.

In an alternative embodiment, a multi-output approach may be used to establish current flow across the spark gap 80. In particular, a high voltage signal that exceeds the breakdown voltage may be provided on the distal defibrillation conductor 68 to initiate the arc phase. Subsequently, a lower voltage therapy signal may be provided on the distal defibrillation conductor 68 while the arc is conducting, allowing a lower voltage therapy signal to be delivered to the distal defibrillation electrode 70.

In some embodiments, one or more additional spark gaps 80 may be connected in series with the distal defibrillation conductor 68 to break the distal defibrillation conductor 68 into segments that are not resonant with the MRI RF fields. The one or more additional spark gaps 80 provide an additional reduction in the amount of MRI-induced energy that is transmitted to the distal defibrillation electrode 70. In some embodiments, the one or more additional spark gaps 80 are arranged periodically along the length of the distal defibrillation conductor 68. This minimizes the energy picked up by the distal defibrillation conductor 68 in an MRI environment.

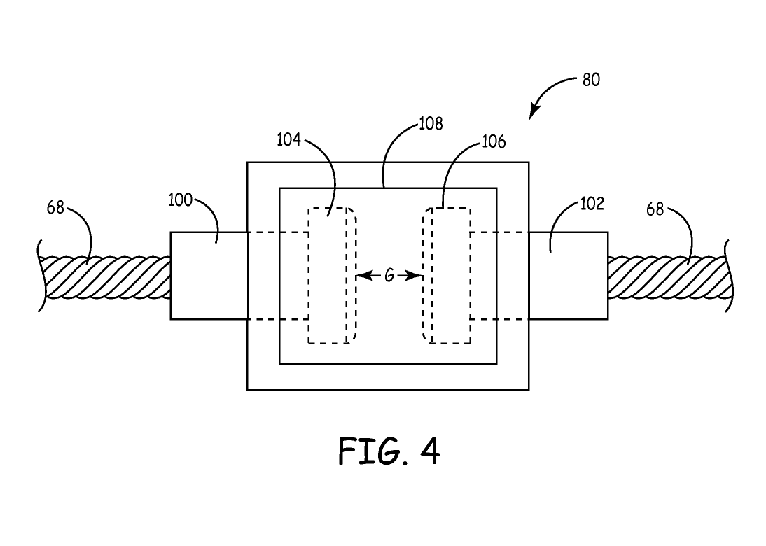

FIG. 4 is a cross-sectional view of an exemplary embodiment of a spark gap 80 connected along distal defibrillation conductor 68. The spark gap 82 that is optionally connected between the proximal defibrillation conductor 72 and the proximal defibrillation electrode 74 may have a similar configuration. The distal defibrillation conductor 68 is connected to a proximal electrode 100 of the spark gap 80 on a proximal side of the spark gap 80 and to a distal electrode 102 on a distal side of the spark gap 80. The distal defibrillation electrode 70 may be connected distally from the portion shown in FIG. 4. While the distal defibrillation conductor 68 is shown as a conductive cable, other configurations are possible (e.g., conductive coil or a wire). In an alternative embodiment, the distal electrode 102 is connected directly to the distal defibrillation electrode 70. In embodiments in which the distal defibrillation conductor 68 includes one or more additional spark gaps along its length, the spark gap 80 illustrated in FIG. 4 may be employed to connect segments of the distal defibrillation conductor 68 in series.

The proximal electrode 100 includes a plate 104 and the distal electrode 102 includes a plate 106. The plates 104, 106 are separated by a gap G, and are surrounded by a medium (e.g., noble gas) within an enclosure 108. As discussed above, the size and shape of the electrode plates 104, 106, the size of the gap G, and the surrounding medium determine the breakdown voltage of the spark gap 80.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical device lead comprising:
a proximal connector configured to couple the lead to a pulse generator;
an insulative lead body extending distally from the proximal connector;
a first lead conductor coupled to the proximal connector and extending through the lead body;
a distal defibrillation electrode; and
a first spark gap connected between the first lead conductor and the distal defibrillation electrode, the first spark gap having a breakdown voltage that prevents transmission of MRI induced signals from the first lead conductor to the distal defibrillation electrode in an MRI environment and allows transmission of therapy signals to the distal defibrillation electrode.

2. The medical device lead of claim 1, and further comprising:
a second lead conductor coupled to the proximal connector and extending through the lead body;
a proximal defibrillation electrode; and
a second spark gap connected between the second lead conductor and the proximal defibrillation electrode, the second spark gap having a breakdown voltage that prevents transmission of MRI induced signals from the second lead conductor to the proximal defibrillation electrode in an MRI environment and allows transmission of a therapy signal to the proximal defibrillation electrode.

3. The medical device lead of claim 1, and further comprising:
one or more additional spark gaps connected along the first lead conductor to break the first lead conductor into segments each having lengths that are non-resonant with RF fields in the MRI environment.

4. The medical device lead of claim 1, wherein breakdown voltage is between about 100 volts (V) and about 150 V.

5. The medical device lead of claim 1, wherein, at voltages below the breakdown voltage, the first spark gap has a resistance greater than about 1,000 megaohms (MΩ) and a capacitance in the range of about 1.0 picofarad (pF) to about 5.0 pF.

6. The medical device lead of claim 1, wherein the first spark gap comprises two electrodes, and wherein the breakdown voltage is a function of a distance between the electrodes.

7. A medical device comprising:
a pulse generator; and
a lead comprising:
a proximal connector that couples the lead to the pulse generator;
an insulative lead body extending distally from the proximal connector;
a first lead conductor coupled to the proximal connector and extending through the lead body;
a distal defibrillation electrode; and
a first spark gap connected between the first lead conductor and the distal defibrillation electrode, the first spark gap having a breakdown voltage that prevents transmission of MRI induced signals from the first lead conductor to the distal defibrillation electrode in an MRI environment and allows transmission of therapy signals to the distal defibrillation electrode.

8. The medical device of claim 7, wherein, to deliver the therapy signal, the pulse generator is programmed to deliver a breakdown signal to the spark gap to establish current flow between the first lead conductor and the distal defibrillation electrode and subsequently provide the therapy signal.

9. The medical device of claim 7, wherein the pulse generator is programmed to disable transmission of therapy signals to the distal defibrillation electrode in the MRI environment.

10. The medical device of claim 7, and further comprising:
a second lead conductor coupled to the proximal connector and extending through the lead body;
a proximal defibrillation electrode; and
a second spark gap connected between the second lead conductor and the proximal defibrillation electrode, the second spark gap having a breakdown voltage that prevents transmission of MRI induced signals from the second lead conductor to the proximal defibrillation electrode in an MRI environment and allows transmission of a therapy signal to the proximal defibrillation electrode.

11. The medical device of claim 7, and further comprising:
one or more additional spark gaps connected along the first lead conductor to break the first lead conductor into segments each having lengths that are non-resonant with RF fields in the MRI environment.

12. The medical device of claim 7, wherein breakdown voltage is between about 100 volts (V) and about 150 V.

13. The medical device of claim 7, wherein, at voltages below the breakdown voltage, the first spark gap has a resistance greater than about 1,000 megaohm (MΩ) and a capacitance in the range of about 1.0 picofarad (pF) to about 5.0 pF.

14. The medical device of claim 7, wherein the first spark gap comprises two electrodes, and wherein the breakdown voltage is a function of a distance between the electrodes.

15. A medical device lead comprising:
a proximal connector configured to couple the lead to a pulse generator;
an insulative lead body extending distally from the proximal connector;
one or more low voltage conductors coupled to the proximal connector and extending through the lead body;
one or more pacing/sensing electrodes each coupled to one of the one or more low voltage conductors;
a proximal defibrillation conductor coupled to the proximal connector and extending through the lead body;
a proximal defibrillation electrode;
a distal defibrillation conductor coupled to the proximal connector and extending through the lead body;
a distal defibrillation electrode; and
a first spark gap connected between the first lead conductor and the distal defibrillation electrode, the first spark gap having a breakdown voltage that prevents transmission of MRI induced signals from the first lead conductor to the distal defibrillation electrode in an MRI environment and allows transmission of therapy signals to the distal defibrillation electrode.

16. The medical device lead of claim 15, and further comprising:
one or more additional spark gaps connected along the first lead conductor to break the first lead conductor into segments each having lengths that are non-resonant with RF fields in the MRI environment.

17. The medical device lead of claim 15, wherein breakdown voltage is between about 100 volts (V) and about 150 V.

18. The medical device lead of claim 15, wherein, at voltages below the breakdown voltage, the first spark gap has a resistance greater than about 1,000 megaohms (MΩ) and a capacitance in the range of about 1.0 picofarad (pF) to about 5.0 pF.

19. The medical device lead of claim 15, wherein the first spark gap comprises two electrodes, and wherein the breakdown voltage is a function of a distance between the electrodes.

20. The medical device lead of claim 15, and further comprising:
a second spark gap connected between the proximal defibrillation conductor and the proximal defibrillation electrode, the second spark gap having a breakdown voltage that prevents transmission of MRI induced signals from the proximal defibrillation conductor to the proximal defibrillation electrode in an MRI environment and allows transmission of a therapy signal to the proximal defibrillation electrode.
